(19) United States
(12) Patent Application Publication
(10) Pub. No.: US 2014/0114281 A1
(43) Pub. Date: Apr. 24, 2014

(54) LAUNCHER FOR ANIMAL TREATMENT

(71) Applicant: Grant Weyer, Fig Tree Pocket, Queensland (AU)

(72) Inventor: Grant Weyer, Fig Tree Pocket, Queensland (AU)

(73) Assignee: SMARTVET PTY LTD, Fig Tree Pocket, Queensland (AU)

(21) Appl. No.: 14/125,333

(22) PCT Filed: Jun. 14, 2012
(86) PCT No.: PCT/AU2012/000684

§ 371 Date: Dec. 11, 2013

Related U.S. Application Data
(60) Provisional application No. 61/497,651, filed on Jun. 16, 2011.

(51) Int. Cl. A61D 7/00; A61M 5/31

(52) U.S. Cl. CPC A61D 7/00; A61M 5/31
USPC 604/506; 604/130

(57) ABSTRACT

A launcher for remotely treating an animal is described. The launcher having a first chamber adapted to receive a first dosage projectile having a first calibre, a second chamber adapted to receive a second dosage projectile having a second caliber smaller than the first calibre, a launcher barrel comprising a first cooperating caliber facilitating launching of the first dosage projectile, an adaptor comprising a second cooperating caliber facilitating launching of the second dosage projectile, and a propulsion assembly for propelling the dosage projectiles. An adaptor is also described, for adapting the launcher to launch dosage projectiles of calibers other than those associated with the launcher barrel.

### FIELD OF THE INVENTION

The present invention relates generally to a launcher for launching dosage projectiles for remote treatment of animals, and an adaptor mountable to a launcher which when mounted to the launcher facilitates the launching of the dosage projectiles. The present invention also relates to methods of remote treatment of animals using dosage projectile launchers.

### BACKGROUND OF THE INVENTION

In practice it is difficult and costly to deliver medicinal compounds to animals, especially if such animals are not kept in enclosures or specifically herded and contained for that purpose. Typically, during outbreaks of disease in wild animals, it is necessary to treat diseased animals by firing one or more injecting darts at the animal in order to deliver a medicinal compound. At other times and under other circumstances it is useful to treat animals with a medical compound which does not penetrate the skin of the animal.

At present, it is necessary to use launchers tailored to launch only one type of projectile when administering treatments to animals. If more than one type of projectile is used, then two different launchers are required.

The present invention provides a new launcher and/or a new launcher adaptor.

### SUMMARY OF THE INVENTION

In a general aspect of the present invention there is provided a launcher for remotely treating animals, the launcher being adapted to receive and launch dosage projectiles having differing calibers.

Preferably the dosage projectiles are launched from the launcher by release of compressed gas.

According to a first aspect of the invention there is provided a launcher for remotely treating an animal comprising:

a chamber adapted to receive a first dosage projectile having a first caliber;

a chamber adapted to receive a second dosage projectile having a second caliber smaller than the first caliber;

a launcher barrel comprising a first cooperating caliber facilitating launching of the first dosage projectile;

an adaptor comprising a second cooperating caliber facilitating launching of the second dosage projectile; and

a propulsion assembly for propelling the dosage projectiles.

Preferably two chambers are provided, wherein a first chamber is adapted to receive the first dosage projectile and the second chamber is adapted to receive the second dosage projectile.

In preferred embodiments the first chamber is disposed at a proximal region of the launcher. In some arrangements the second chamber is disposed inside or adjacent or abutting the first chamber.

In preferred embodiments the second chamber is longitudinally spaced from the first chamber. Preferably the second chamber is disposed adjacent a distal region of the launcher.

Preferably the adaptor includes an adaptor barrel.

Preferably the adaptor also includes a connector so as to connect the adaptor barrel to a portion of the launcher. Preferably the connector sealingly connects to the launcher so as to inhibit egress of air or gas from a connecting region of the barrel during launch of the projectile. Preferably the adaptor and/or the connector includes a seal for sealing the adaptor barrel to the launcher.

In some arrangements the connector is disposed in an intermediate region of the adaptor barrel. Preferably the connector is disposed at a proximal end of the adaptor. Preferably the connector connects to the launcher at a distal end of the launcher barrel.

Preferably the adaptor barrel extends from the launcher barrel when the adaptor barrel is connected to the launcher barrel.

The connector may include a thread for connecting to a corresponding connecting region of the launcher and the cooperating portion of the launcher is a cooperating thread portion on the muzzle of the launcher barrel.

In some arrangements the adaptor in use is connected to the launcher so that it extends into the launcher barrel such that a proximal end of the adaptor is disposed adjacent to or abutting the first chamber.

In preferred arrangements the adaptor and second chamber are integral with one another, and the second chamber or adaptor chamber is disposed at a proximal end of the adaptor barrel body. Preferably the second chamber is disposed, when the adaptor is connected, adjacent the muzzle of the launcher.

Preferably the connector includes a locator to locate the barrel body relative to the launcher barrel. Preferably the locator is a rib for cooperating with a cooperating barrel groove on the muzzle of the launcher barrel. Preferably the locator is a thread end of the connecting thread.

The adaptor chamber may include a breech cover so as to cover the chamber. Preferably the breech cover includes a seal to seal the adaptor chamber. Preferably the breech cover is adapted to move between an open position and a closed position.

Preferably the breech cover includes an indicating portion to indicate to a user whether the breech cover is in the open or closed position. The breech cover may hinge or slide between the open and closed positions. The hinge may be disposed at a distal or muzzle end of the breech cover. Preferably, the breech cover rotates between the open and closed positions. A detent may be disposed to hold the breech cover in the closed and open positions.

A safety interlock may be provided to inhibit firing of the dosage dart unless the adaptor breech cover is in the closed position.

The adaptor may include a sight for facilitating aim of the launcher in use.

In some arrangements the dosage projectiles which the launcher for remotely treating animals is adapted to launch include substantially spherical dosage projectiles and dosage darts. Preferably the dosage projectiles of the first caliber are spherical dosage projectiles and the dosage projectiles of the second, smaller caliber are dosage darts.

Examples of substantially spherical dosage projectiles systemic application of an active agent to an animal suitable for the present invention can be found in WO 2008/052263 (Smartvet Pty Ltd), incorporated herein in its entirety by reference. Although the spherical dosage projectiles of WO 2008/052263 contain a transdermal delivery agent, it will be appreciated that spherical dosage projectiles may be formulated without a transdermal delivery agent for topical application to an animal.

Examples of substantially spherical dosage projectiles configured for consumption by an animal to suitable for the present invention can be found in WO 2012/034167 (Smartvet Pty Ltd), incorporated herein in its entirety by reference.

Examples of dosage darts suitable for the present invention can be found in U.S. Pat. No. 7,795,263.

The dosage projectiles typically contain one or biologically active agents for administering to an animal.

The launcher can be used to launch dosage projectiles for remotely treating an animal. The launcher is suitable for use with a wildlife or game animal such as deer or other antelope species or buffalo, commercial or production livestock such as cattle and horses or larger bodied feral animals such as horses, buffalo, wild dogs, pigs, and the like.

A hopper may be provided to deliver the spherical dosage projectiles to the chamber or the spherical dosage projectile may be inserted manually.

A magazine may be provided to deliver dosage darts to the chamber or the darts may be inserted manually.

According to a second aspect of the invention, there is provided a launcher for remotely treating an animal comprising:

a first chamber disposed at a proximal region of the launcher, the chamber adapted to receive a first dosage projectile having a first caliber;

a launcher barrel comprising a first cooperating caliber facilitating launching of the first dosage projectile;

a propulsion assembly for propelling the dosage projectiles; and

an adaptor comprising an adaptor barrel having a second caliber adapted to cooperate with a second dosage projectile having a caliber smaller than the first caliber, the adaptor including a second chamber disposed at a proximal end of the adaptor barrel, the second chamber being configured to receive the second dosage projectile, the proximal end of the adaptor barrel being detachably sealed to and connected to a muzzle end of the launcher barrel so that the adaptor extends away from the muzzle end of the launcher barrel, the adaptor including a breech cover to seal and cover the chamber in a covered position.

According to a third aspect of the invention, there is provided an adaptor to facilitate launching dosage projectiles of differing calibres from a launcher, the launcher having a launcher barrel of a first selected caliber of a size adapted to cooperate with dosage projectiles of a first caliber, and a propulsion assembly for propelling the dosage projectiles, the adaptor comprising:

an adaptor barrel having a second cooperating caliber being smaller than the launcher barrel cooperating caliber, the adaptor barrel adapted to cooperate with dosage projectiles of the second caliber;

the adaptor barrel adapted to connect to a portion of the launcher; and

a breech operatively connected to or integral with the adaptor barrel for receiving a dosage projectile of the second calibre.

Preferably, the adaptor includes a breech cover to seal and cover the chamber of the adaptor barrel in a covered position.

Preferably, the adaptor barrel includes a connector for connecting to a cooperating portion of the launcher.

According to a fourth aspect of the invention, there is provided a method for adapting a launcher for launching dosage projectiles of a first selected caliber to launch dosage projectiles of a second, smaller caliber, the launcher having a launcher barrel of a cooperating first caliber and a propulsion assembly for propelling the dosage projectiles, the method comprising the step of:

attaching an adaptor according to the second aspect of the present invention to the launcher, so that the launcher can launch the second dosage projectile having a second caliber smaller than the first caliber.

According to a fifth aspect of the invention, there is provided a method for remotely treating an animal comprising launching a dosage projectile to an animal from a launcher according to the first aspect of the present invention.

Preferably the animal is a livestock animal or a feral animal.

### BRIEF DESCRIPTION OF THE DRAWINGS

The invention will now be further explained and illustrated by reference to the accompanying drawings in which:

FIG. 1 is an isometric view of a launcher according to a preferred embodiment of the invention, the launcher having a breech cover in an open position; as well as an isometric view of a dosage dart for firing by the launcher;

FIG. 2 is an isometric view of the launcher of FIG. 1 showing the breech cover in the closed position; and

FIG. 3 is an isometric view of an adaptor shown as part of the launcher of FIG. 1, the adaptor also according to a preferred embodiment of the present invention, showing the breech cover in the open position.

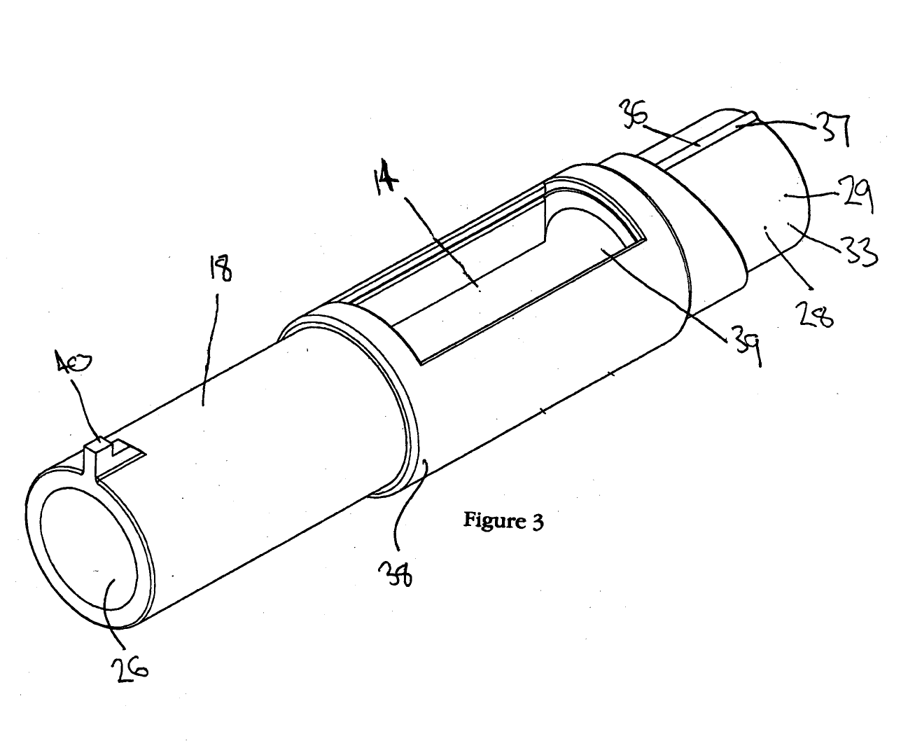

### DETAILED DESCRIPTION OF A PREFERRED EMBODIMENT

Referring to the drawings there is shown a launcher for remotely treating an animal generally indicated at 10. The launcher 10 comprises a first chamber 12 adapted to receive a first dosage projectile having a first caliber, and a second chamber 14 adapted to receive a second dosage projectile having a second caliber smaller than the first caliber.

The launcher 10 includes a launcher barrel 16 having a cooperating first caliber sized to cooperate with the first dosage projectile and an adaptor 18 comprising a cooperating second caliber for cooperating with the second dosage projectile. A propulsion assembly disposed at 20 is provided for propelling the dosage projectiles, one example of which is shown at 22.

The dosage projectiles which the launcher 10 is adapted to launch include . substantially spherical dosage projectiles (not shown) and dosage darts 22. The dosage projectiles of the first caliber are spherical dosage projectiles and the dosage projectiles of the second, smaller caliber are dosage darts 22.

Examples of substantially spherical dosage projectiles systemic application of an active agent to an animal suitable for the present invention can be found in WO 2008/052263 (Smartvet Pty Ltd), incorporated herein in its entirety by reference. Although the spherical dosage projectiles of WO 2008/052263 contain a transdermal delivery agent, it will be appreciated that spherical dosage projectiles may be formulated without a transdermal delivery agent for topical application to an animal,

Examples of substantially spherical dosage projectiles configured for consumption by an animal to suitable for the present invention can be found in WO 2012/034167 (Smartvet Pty Ltd), incorporated herein in its entirety by reference.

Examples of dosage darts suitable for the present invention can be found in U.S. Pat. No. 7,795,263. Dosage darts are generally of a smaller diameter than the spherical dosage projectiles.

While spherical dosage projectiles are configured to impact on an animal or be consumed by an animal to provide a biologically active agent, dosage darts are configured to break the skin barrier to inject a biologically active agent. Having one launcher that can be used to project or launch spherical dosage projectiles and also dosage darts as required is particularly useful.

Large non-domesticated mammals are typically anesthetized and immobilized for reasons of medical examination or treatment, or for reasons of physiologic or morphologic study or herd health assessment. Any such examination or study is impossible unless the animals are immobilized, because the non-domesticated nature of the animals makes it impossible to approach or safely handle them, even in captive environments such as zoological parks or animal preserves. Performing any procedure that may produce pain is also impossible unless the animals are anesthetized, because the animals will resist such procedures, even if the animals are substantially immobilized.

Because non-domesticated animals cannot be approached for injection by hand, it is necessary to remotely deliver an anesthetizing and immobilizing drug, typically from a single dart projected or shot from a gun at the distance which the animal will tolerate before fleeing, i.e. the flight distance. A dosage dart is a syringe-like structure which has a drug-confining chamber and an attached hypodermic needle. When the dart impacts the animal, the hypodermic needle penetrates through the hair and hide of the animal and into muscle. An expulsion chamber containing compressed gas or a small explosive charge is activated upon impact and quickly forces the liquid anesthetizing and immobilizing drug from the drug-confining chamber through the needle into the muscle of the animal. The drug enters the bloodstream, and over a short time period anesthetizes and immobilizes the animal.

The flight distance which a non-domesticated animal will tolerate varies with the species and the environment. To accommodate lengthy flight distances, the dart should have a trajectory distance of up to about 70 meters with good accuracy. Any shorter range will not always be satisfactory because it is usually difficult or impossible to approach free ranging wildlife species any closer than approximately 35 meters. The trajectory range should permit sufficient accuracy to deliver the dart into the muscle mass of a shoulder or hind quarter of the animal, because impacting the dart with a bony area such as the rib cage or lower extremity will not allow the needle to penetrate adequately to deliver the full dose of the anesthetizing and immobilizing drug.

Increasing the velocity of a dart will increase its range and will contribute to its accuracy, but increased velocity of the dart increases the risk of excessive trauma or tissue wound to the animal from the impact of the dart. The risk of such a tissue wound is directly related to the kinetic energy of the dart, and the kinetic energy increases with the square of the velocity.

The weight of the dart also affects range and accuracy. A heavier weight dart will have less effective range than a lighter weight dart. The accuracy in placement of a heavier weight dart may be less than to a lighter weight dart, because the heavier dart may spend more time in trajectory and therefore fall more under the influence of gravity. A heavier dart will also create more tissue injury or trauma upon impact, resulting in more risk of disease from bacteria growth, as described above. The amount of tissue injury or trauma increases linearly with weight, rather than in relation to the square of the velocity.

One influence on the weight of the dart is the volume of the immobilizing and anesthetizing drug that it must carry. Larger volumes of the drug are heavier and require a larger dart. The weight and species of the non-domesticated animal contributes to the amount or volume of the immobilizing and anesthetizing drug that must be used. Although darts with a maximum capacity of about 5 or more ml are available, such darts can be susceptible to limited range and accuracy and excessive tissue injury. A dart with a drug-carrying capacity of 3 ml or less can also be used for the present invention.

The first chamber 12 is adapted to receive the first, larger-caliber dosage projectile and the second chamber 14 is adapted to receive the second, smaller-caliber dosage projectile 22.

The first chamber 12 is disposed at a proximal end region 24 of the launcher 10.

The second chamber 14 is spaced from the first chamber along a barrel or longitudinal axis and is disposed adjacent a distal end 25 of the launcher 10.

The adaptor 18 includes an adaptor barrel 26, its caliber cooperating with that of the second dosage projectile. The adaptor 18 also includes a connector 28 so as to connect to a portion of the launcher 10. The connector 28 sealingly connects to the launcher 10 so as to inhibit egress of air from a connecting and/or a chamber region of the barrel 16 during launch of the projectile 22. Preferably the adaptor 18 includes a seal 30 for sealingly connecting to the launcher 10.

The connector 28 is disposed at a proximal end 29 of the adaptor 18. The connector 28 connects to the launcher barrel 16. The connector 28 includes a thread 33 for connecting to a corresponding connecting region 34 of the launcher 10 and the cooperating portion of the launcher is a cooperating thread portion (not shown) on the muzzle 35 of the launcher barrel 16.

The adaptor 18 and second chamber 14 are integral with one another, and the second chamber or adaptor chamber 14 is disposed at the proximal end 29 of the adaptor barrel body 18. The second chamber 14 is disposed, when the adaptor 18 is connected to the launcher 10, adjacent the muzzle 35 of the launcher 10.

The connector includes a locator 36 to locate the barrel body relative to the launcher barrel 16. The locator is a rib 37 for cooperating with a cooperating barrel groove (not shown) on the muzzle 35 of the launcher barrel 16.

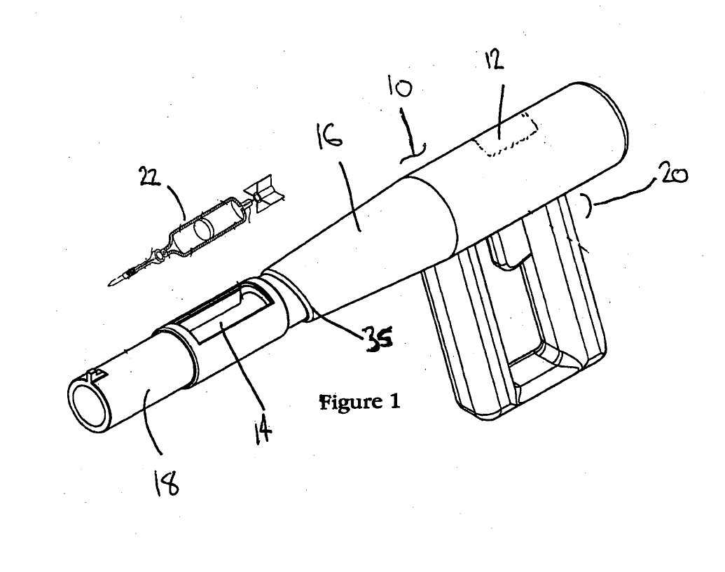

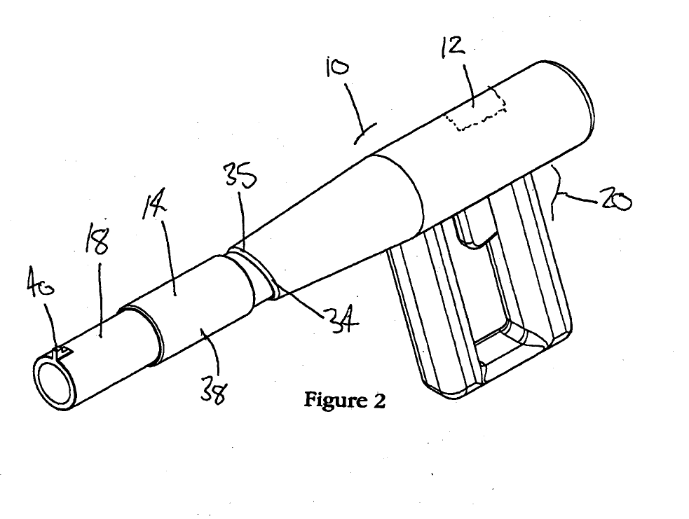

The adaptor chamber 14 includes a breech cover 38 so as to cover the chamber 14. The breech cover 38 includes a seal 39 to seal the adaptor chamber 14. The breech cover can move between an open position (FIG. 1) and a closed position (FIG. 2). The breech cover includes an indicating portion (not shown) to indicate to a user whether the breech cover is in the open or closed position. The breech cover 38 rotates between the open and closed positions.

A safety interlock (not shown) is provided to inhibit firing of the dosage dart unless the adaptor breech cover is in the closed position.

A hopper (not shown) is provided to deliver the spherical dosage projectiles to the chamber.

A magazine (not shown) is provided to deliver dosage darts to the chamber 14 or the darts may be inserted manually.

Other portions of the launcher 10 are conventional and in use it operates so as to launch spherical projectiles of larger caliber as normal, that is, when the adaptor 18 is not connected to the muzzle 35. The launcher 10 operates to launch dosage darts 22 of smaller caliber when the adaptor 18 is connected to the muzzle 35. The operating principle in the latter case is that the adaptor 18 seals the end of the muzzle 35 so that the only opening between the propulsion assembly 20 and the muzzle end of the adaptor is the adaptor body bore itself. So, when the trigger is actuated, the high pressure gas from the propulsion assembly 20 is sent along the barrel 16 to launch the dosage dart 22 from the breech 14 out the muzzle end of the adaptor 14 and towards an animal.

That is, to launch a dosage projectile from the barrel of the launcher 10, the propulsion assembly 20 is used. Air or gas needed for the launcher 10 can come from a compressed air or gas tank (not shown). Suitable tanks can be filled with nitrogen, CO₂ and/or compressed air.

A valved tube can be located in the barrel 16 which extends past a spring, bolt and hammer to the air inlet valve (not shown). A tank of compressed air is connected to the gas inlet valve to provide the energy needed to launch the dosage projectile. When an actuator is cocked, a dosage projectile is advanced into the breech 12 or 14 from a hopper or suitable loader such as for example a magazine (not shown). The spring is released when the trigger is pulled allowing a burst of air from the tank to pass through the inlet valve and launch the dosage projectile through the barrel to the animal (not shown).

A compressed air tank can be filled by being attached to a larger tank that supplies the air or gas.

Examples of suitable propulsion systems and applicators used in the paintball and air gun industries and suitable for the present invention can be found in U.S. Pat. No. 6,990,971, US 2010/0051008 and U.S. Pat. No. 5,823,173. It will be appreciated that spring loaded air compression arrangements can also be used for the propulsion system.

The adaptor can include a sight 40 for facilitating aim of the launcher 10.

The launcher is particularly suitable for remotely treating an animal. The launcher is suitable for use with a wildlife or game animal such as deer or other antelope species or buffalo, commercial or production livestock such as cattle and horses or larger bodied feral animals such as horses, buffalo wild dogs, pigs, and the like.

The animal can treated for a condition such as disease, parasite infestation or condition, dietary deficiency, or fertility. In particular, the condition is insect or parasite infection or infestation.

The dosage projectiles can contain one or more biologically active agents for treating an animal.

The biologically active agent is typically present at a concentration (% v/v) of from about 0.1% to 20%. It will be appreciated that the concentration of the biologically active agent will be related to the dosage required for a particular size of animal.

The biologically active agent can be a pharmacological agent such as a veterinary pharmaceutical for treating insect pests or disease.

The biologically active agent can be a health supplement such as a vitamin or mineral.

The biologically active agent can be a vaccine or immunogenic compound. The biologically active agent may also include protein-based agents, such as crude or purified cell lysates, sub-unit vaccines, protein-based antigen display systems, antigens, peptides, oligopeptides, or polypeptides.

The biologically active agent may include drugs such as contraceptives, analgesics, anti-inflammatories, vasodilators, bronchodilators, diuretics, anti-histamines, tranquilizers, anti-fungals, vitamins, muscle relaxants, and anti-virals, anti-parasitic compositions, anthelmintics, acaricides, insecticides, and the like.

The biologically active agent may include a hormone such as a progesterone, estrogen, testosterone, derivatives thereof, and/or combinations of such hormones.

The biologically active agent may be poison or toxin used to kill an animal follow administration or consumption.

In this specification, where a document, act or item of knowledge is referred to or discussed, this reference or discussion is not an admission that the document, act or item of knowledge or any combination thereof was at the priority date:

part of common general knowledge; or

known to be relevant to an attempt to solve any problem with which this specification is concerned.

The word ‘comprising’ and forms of the word ‘comprising’ as used in this description do not limit the invention claimed to exclude any variants or additions.

Modifications and improvements to the invention will be readily apparent to those skilled in the art. Such modifications and improvements are intended to be within the scope of this invention.

1. A launcher for remotely treating an animal comprising:
a first chamber adapted to receive a first dosage projectile having a first caliber;
a second chamber adapted to receive a second dosage projectile having a second caliber smaller than the first caliber;
a launcher barrel comprising a first cooperating caliber facilitating launching of the first dosage projectile;
an adaptor comprising a second cooperating caliber facilitating launching of the second dosage projectile; and
a propulsion assembly for propelling the dosage projectiles.

2. The launcher in accordance with claim 1 wherein the first chamber is disposed at a proximal region of the launcher and the second chamber is longitudinally spaced from the first chamber.

3. The launcher of claim 1 wherein the adaptor includes an adaptor barrel.

4. The launcher in accordance with claim 3 wherein the adaptor includes a connector so as to connect the adaptor barrel to a portion of the launcher.

5. The launcher in accordance with claim 4 wherein the connector is disposed at a proximal end of the adaptor barrel.

6. The launcher in accordance with claim 5 including a seal for sealing the adaptor barrel to the launcher barrel.

7. The launcher of claim 4 wherein the connector connects to the launcher at a distal end of the launcher barrel.

8. The launcher of claim 3 wherein the adaptor barrel extends from the launcher barrel when the adaptor barrel is connected to the launcher barrel.

9. The launcher of claim 8 wherein the connector includes a connecting thread for connecting to a connecting region of the launcher.

10. The launcher of claim 1 wherein the adaptor and second chamber are integral with one another.

11. The launcher of claim 3 wherein the second chamber is disposed at a proximal end of the adaptor barrel.

12. The launcher of claim 4 wherein the connector includes a locator to locate the adaptor barrel relative to the launcher barrel.

13. The launcher in accordance with claim 12 wherein the locator is a rib for cooperating with a cooperating barrel groove on the muzzle of the launcher barrel.

14. The launcher of claim 10 wherein the adaptor chamber includes a breech cover so as to cover the second chamber.

15. The launcher in accordance with claim 14 wherein the breech cover includes a seal to seal the adaptor chamber.

16. The launcher in accordance claim 15 wherein the breech cover is adapted to slide around the adaptor barrel between an open position and a closed position.

17. The launcher of claim 14 wherein a detent is provided to hold the breech cover in either of the closed or open positions.

18. The launcher of claim 14 wherein a safety interlock is provided to inhibit firing of the dosage projectile unless the adaptor breech cover is in the closed position.

19. The launcher claim 1 wherein a hopper is provided to deliver the first dosage projectiles to the first chamber.

20. The launcher of claim 1 wherein a magazine is provided to deliver dosage projectiles to the chamber.

21. The launcher of claim 1 wherein the first dosage projectile is a spherical dosage projectile and the second dosage projectile is a dosage dart.

22. A method for remotely treating an animal comprising launching a dosage projectile to an animal from a launcher in accordance with claim 1.
